(19) United States
(12) Patent Application Publication
(10) Pub. No.: US 2011/0219675 A1
(43) Pub. Date: Sep. 15, 2011

(54) ENZYMATIC PRODUCTION OF FATTY ACID ETHYL ESTERS

(75) Inventors: Jesper Brask, Bagsvaerd (DK); Per Munk Nielsen, Hilleroed (DK); Yuan Xu, Bagsvaerd (DK)

(73) Assignee: Novozymes A/S, Bagsvaerd (DK)

(21) Appl. No.: 13/125,189

(22) PCT Filed: Oct. 29, 2009
(86) PCT No.: PCT/EP2009/064300

§ 371 Date: Apr. 20, 2011

Related U.S. Application Data
(60) Provisional application No. 61/110,155, filed on Oct. 31, 2008.

(30) Foreign Application Priority Data
Oct. 31, 2008 (EP) 08168052.2

(51) Int. Cl. C10L 1/02; C12P 7/64

(52) U.S. Cl. 44/388; 435/134; 44/389

(57) ABSTRACT

The invention relates to a method of producing fatty acid ethyl esters comprising: a) reacting a substrate comprising triglycerides, diglycerides, monoglycerides, free fatty acids, or any combination thereof, with at least one immobilized lipolytenzyme, to provide a reaction mixture wherein the enzyme loading is below 30% w/w with respect to the substrate, and the molar ratio of ethanol to fatty acid (EtOH:FA) is at least 3.0 equivalents; b) separating the immobilized lipolytic enzyme from the resulting reaction mixture; and c) subjecting the immobilized lipolytic enzyme to at least one further reaction directly without modifications.

### BACKGROUND OF THE INVENTION

1. Field of the Invention

The present invention relates to enzymatic production of fatty acid ethyl esters. The invention particularly relates to the activity of immobilized enzymes for re-use in synthesis of fatty acid ethyl esters and the effect of ethanol excess on enzyme activity.

2. Description of the Related Art

Enzymatic processing of oils and fats for biodiesel is technically feasible. Biodiesel produced by enzymatic bioconversion is, compared with chemical conversion, more environmental friendly and therefore desirable. However, with very few exceptions, enzyme technology is not currently used in commercial scale biodiesel production. This is mainly due to non-optimized process design and a lack of available cost effective enzymes. The technology to re-use enzymes has typically proven insufficient for the processes to be competitive.

Lipases catalyze the transesterification of a triglyceride substrate with alcohols such as methanol (MeOH) and ethanol (EtOH) to form fatty acid alkyl esters such as fatty acid methyl esters (FAME) and fatty acid ethyl esters (FAEE) respectively. A problem with such enzyme catalyzed processes is that the lipase may be inactivated by the alcohol. Therefore, the concentration of alcohol is generally kept low throughout the process. The alcohol tolerance is influenced by factors such as the enzyme, the alcohol, the way the enzyme is immobilized, etc. In general, the smaller the alcohol, the more inactivating it is. Hence MeOH is more inactivating than EtOH, which is more inactivating than propanol (PrOH), etc. (“Enzymatic biodiesel production: Technical and economical considerations” Nielsen, P M. et al. (2008) Eur. J. Lipid Sci. Technol., vol. 110, p. 692-700).

The main obstacle for full exploitation of lipolytic enzymes in the production of biodiesel is the cost. Therefore, re-use of lipolytic enzymes is essential from an economic point of view, which may be achieved by using lipolytic enzymes in an immobilized form. Methods in which immobilized lipolytic enzymes are re-used in the production of biodiesel have been described, some of which are mentioned below:

“Different enzyme requirements for the synthesis of biodiesel: Novozym® 435 and Lipozyme® TL IM” Hernandez-Martin, E et al. (2008) Bioresource Technology vol. 99, p. 277-286. describes the conversion of different vegetable oils by Novozym 435, Lipozyme TL IM and Lipozyme RM IM in the production of fatty acid ethyl esters. Re-use of Novozyme 435 was demonstrated using an enzyme loading of 50% w/w with respect to the substrate in a 7 hour reaction at 25° C. where Novozyme 435 was washed with chloroform and dried between each reaction cycle. Re-use of Lipozyme TL IM was likewise attempted with 10% enzyme, 24 h reaction time, and chloroform wash between reaction cycles. A EtOH/FA ratio of 1 (1 eq) was used in the reaction. However, the authors found that the enzyme had only 10% residual activity already after the 1^{st} cycle.

“Selective enzymatic synthesis of lower acylglycerols rich in polyunsaturated fatty acids” Hernandez-Martin, E et al. (2008) Eur. J. Lipid Sci. Technol. Vol. 110, p. 325-333 describes the conversion of soybean oil by Novozyme 435 in the production of fatty acid ethyl esters. Re-use of Novozyme 435 was demonstrated using an enzyme loading at 50% w/w with respect to the substrate in a 1 hour reaction at 25° C. where Novozyme 435 was washed either in chloroform or 2-propanol and subsequently dried between each reaction cycle.

“Improved enzyme stability in lipase-catalysed synthesis of fatty acid ethyl ester from soybean oil” Rodrigues, R C et al. (2008) Appl. Biochem. Biotechnol. Vol. 152, p. 394-404 describes the conversion of soybean oil by Lipozyme TL IM in the production of fatty acid ethyl esters. Re-use of Lipozyme TL IM was demonstrated using an enzyme loading at 25% w/w with respect to the substrate in a 12 hour reaction at 26° C. in the presence of 4% w/w added water with respect to the substrate. Lipozyme TL IM was washed in hexane, water, EtOH or propanol and subsequently dried for 24 hours at 40° C. between each reaction cycle. Best reusability was found with hexane wash. Without a washing step, enzyme activity quickly declined. EtOH to oil ratio was 7.5:1, meaning 2.5 eq relative to fatty acids. Reusability was not tested with higher EtOH amounts.

“Immobilized Pseudomonas cepacia lipase for biodiesel fuel production from soybean oil” Noureddini, H et al. (2005) Bioresource Technology vol. 96, p 769-777. describes conversion of soybean oil for the production of fatty acid ethyl esters in the presence of high amounts of alcohol. The reaction was catalyzed by Pseudomonas cepacia (PS) lipase immobilized in a hydrophobic sol-gel matrix. Re-use of the immobilized PS lipase was demonstrated using an enzyme loading at 30% w/w with respect to the substrate in a 1 hour reaction in the presence of 0.3 g corresponding to 3% w/w added water with respect to the substrate.

“Conversion of acid oil by-produced in vegetable oil refining to biodiesel fuel by immobilized Candida antarctica lipase” Watanabe, Y et al. (2007) Journal of Molecular Catalysis vol. 44, p. 99-105 describes a two-step reaction for production of FAME from acid oil in the presence of a molar ratio of 1-10 MeOH:FA using glycerol activated Candida antarctica lipase immobilized on a hydrophobic carrier material (Novozym 435).

Methods for producing fatty acid ethyl esters are presently based on relatively high enzyme loadings which for industrial purposes are undesirable. Further, most applications rely on the enzyme being immobilized on a hydrophobic support material (e.g. Novozym 435). The hydrophobic polymeric materials are in general more costly than inorganic hydrophilic materials (e.g. silica). Modification such as a step of washing and drying the immobilized lipolytic enzyme between each reaction cycle is currently comprised in most methods, and furthermore, addition of various amounts of water to the reaction is also comprised in many methods reported.

Thus, there is still a need to develop improved methods wherein lipolytic enzymes, immobilized on low-cost hydrophilic support materials, may be re-used in production of fatty acid ethyl esters.

### SUMMARY OF THE INVENTION

The inventors have surprisingly found that lipolytic enzymes immobilized on a hydrophilic carrier material in the presence of high amounts of ethanol may efficiently be re-used for production of fatty acid ethyl esters.

In a first aspect the invention relates to a method of producing fatty acid ethyl esters comprising:
a) reacting a substrate comprising triglycerides, diglycerides, monoglycerides, free fatty acids, or any combination thereof, with at least one immobilized lipolytic enzyme, to provide a reaction mixture wherein the enzyme loading is below 30% w/w with respect to the substrate, and the molar ratio of ethanol to fatty acid (EtOH:FA) is at least 3.0 equivalents;
b) separating the immobilized lipolytic enzyme from the resulting reaction mixture; and
c) subjecting the immobilized lipolytic enzyme to at least one further reaction directly without modifications.

In a second aspect the invention relates to re-use of at least one immobilized lipolytic enzyme in the production of fatty acid ethyl esters obtained by reacting ethanol with a substrate comprising triglyceride, diglyceride, monoglyceride; free fatty acids or any combination thereof, wherein the molar ratio of ethanol to fatty acid in the substrate (EtOH:FA) is at least 3.0 equivalents; the enzyme loading is below 30% w/w with respect to the substrate; and which enzyme after use in a conversion reaction is separated from the resulting reaction mixture and reused directly without modifications in the next conversion reaction.

In a third aspect the invention relates to a composition obtained by the method wherein said composition comprises at least two of the following components selected from the group containing: fatty acid ethyl esters; triglyceride; diglyceride; monoglyceride; glycerol; and water.

In a fourth aspect the invention relates to use of the composition obtained by the method as fuel.

In a fifth aspect the invention relates to a fuel comprising the composition obtained by the method.

### BRIEF DESCRIPTION OF THE FIGURE

FIG. 1 shows re-use of immobilize Thermomyces lanuginosa lipase for synthesis of fatty acid ethyl esters using 1-6 eq. of ethanol (EtOH). Labels refer to “cycle number”, “eq. EtOH”. Hence, “1,1” means “1^{st} cycle, 1 eq. EtOH”, while “2.3” means “2^{nd} cycle, 3 eq. EtOH”. The white/gray bars refer to fatty acid ethyl esters content (%, w/w) after 4 h reaction, while the black bars refer to fatty acid ethyl esters content after 24 h reaction. It is evident that 1 eq. and 2 eq. EtOH result in very little fatty acid ethyl esters formation in the 2^{nd} and following cycles. For more details, please refer to Example 1.

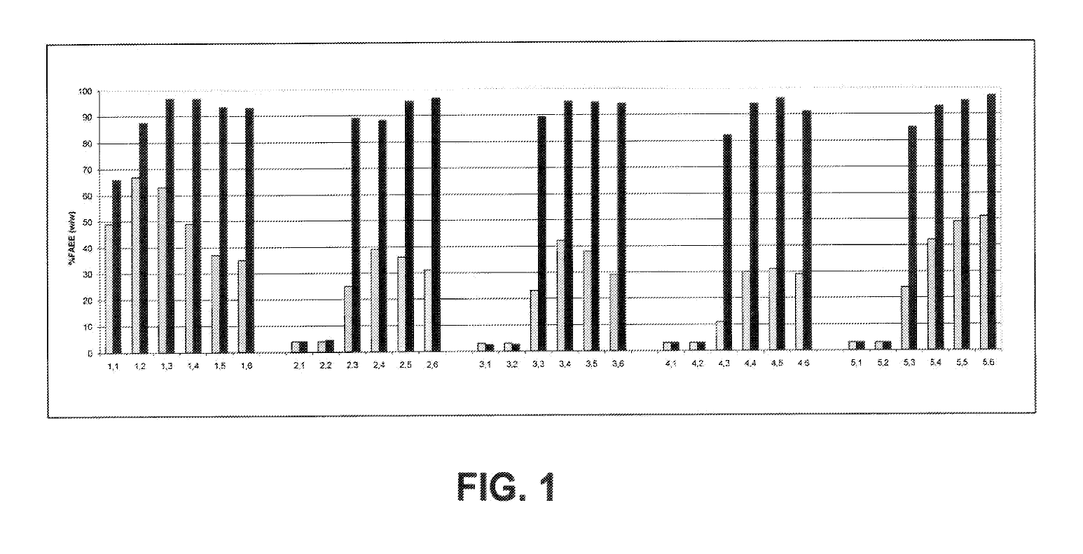

### DEFINITIONS

Biodiesel: The term “biodiesel” is defined herein as fatty acid alkyl esters of short-chain alcohols obtained by the following reaction: Glycerides+FFA+alcohol→fatty acid alkyl ester (biodiesel)+glycerol+water, where a short-chain alcohol is an alcohol having 1 to 5 carbon atoms (C₁-C₅).

Lipolytic enzyme: The term “lipolytic enzyme” is defined herein as a triacylglycerol acylhydrolase, EC 3.1.1.3 that catalyzes reactions such as hydrolysis, interesterification, transesterefication, esterification, alcoholysis, acidolysis and aminolysis.

Substrate: The term “substrate” is defined herein as a substrate comprising triglyceride, diglyceride, monoglyceride, free fatty acid or any combination thereof.

### DETAILED DESCRIPTION OF THE INVENTION

Biodiesel represents a promising alternative fuel for use in compression-ignition (diesel) engines. The biodiesel standards (DIN 51606, EN 14214, and ASTM D6751) require or indirectly specify that biodiesel should be fatty acid methyl esters (FAME). However, we will use the term biodiesel broadly for fatty acid alkyl esters of short-chain alcohols obtained by the following reaction: Glycerides+FFA+alcohol→fatty acid alkyl ester (biodiesel)+glycerol+water. A short-chain alcohol is an alcohol having 1 to 5 carbon atoms (C₁-C₅). A preferred short-chain alcohol is ethanol.

### Destabilizing Effect of Alcohols

Immobilized lipolytic enzymes are in general rather thermostable in oils, and the commercial process for enzymatic interesterification is generally performed at 70° C. Short-chain alcohols, however, have a negative impact on the stability and accordingly the activity of lipolytic enzymes and this destabilizing effect increases with increasing temperature. The destabilizing effect of alcohols on lipolytic enzymes seems to decrease with increasing alcohol molecular weight. The connection between solubility of the alcohol in oil and the destabilizing effect of the oil has been noted by several groups.

A few cases have described a positive effect of high alcohol dosage: In situations were the enzyme is very robust or if a larger alcohol without inactivating properties is used inactivation is not a problem. In that case the high alcohol concentration may be an advantage to drive the equilibrium reaction to full conversion.

Full conversion of a triglyceride-substrate results in formation of glycerol as a byproduct. Glycerol has been shown to inactivate immobilized enzymes, presumably by physically blocking the access of substrate to the enzyme. It has been suggested that high alcohol concentrations may help avoiding that glycerol inactivate immobilized enzymes by keeping the glycerol in solution. It has been shown that adsorbed glycerol on used silica particles may be removed by ethanol followed by drying (“Near-quantitative production of fatty acid alkyl esters by lipase-catalyzed alcoholysis of fats and oils with adsorption of glycerol by silica gel” Stevenson et al. (1994) Enzyme Microb. Technol., vol. 16, p. 478-484).

Methods in which immobilized lipolytic enzymes are re-used in the production of biodiesel in the presence of large excess of ethanol have so far not been successful or industrial attractive.

It is therefore surprising that fatty acid ethyl esters may be produced in the presence of at least 3.0 equivalents, a relatively high molar ratio of ethanol to fatty acid in the substrate (EtOH:FA) as disclosed in the present invention and illustrated by the examples.

It has repeatedly been pointed out that the presence of water is important to maintain the activity of the lipolytic enzyme, and the majority of currently known methods prescribe addition of water to the reaction. It has surprisingly been found that the method of the present invention may be performed without additional water.

Steps of washing and drying have often been included in methods known in the art for the purpose of removing in particular glycerol which is considered to inhibit the activity of the lipolytic enzyme. Inclusion of a washing step using two different solvents, hexane or tert-butanol (t-BuOH) surprisingly showed that washing did not change the enzyme activity over at least 3 reaction cycles when using at molar ratio of 2.0 eq. EtOH:FA (Table 2) or over at least 10 cycles when using at molar ratio of 3.5 eq. EtOH:FA (Table 3) in comparison with no washing as shown in example 2.

In certain embodiments the present invention relates to a method of producing fatty acid ethyl esters comprising: a) reacting a substrate comprising triglycerides, diglycerides, monoglycerides, free fatty acids, or any combination thereof, with at least one immobilized lipolytic enzyme, to provide a reaction mixture wherein the enzyme loading is below 30% w/w with respect to the substrate, and the molar ratio of ethanol to fatty acid (EtOH:FA) is at least 3.0 equivalents; b) separating the immobilized lipolytic enzyme from the resulting reaction mixture; and c) subjecting the immobilized lipolytic enzyme to at least one further reaction directly without modifications.

### Lipolytic Enzymes

Most lipolytic enzymes used as catalysts in organic synthesis are of microbial and fungal origin, and these are readily available by fermentation and basic purification. Lipolytic enzymes extracted from various sources have successfully been used in the production of biodiesel. Candida Antarctica B lipase immobilized on hydrophobic acrylic resin (Novozym 435) has been the most commonly used enzyme for the production of biodiesel. However, depending on experimental variables such as substrate, alcohol, water, temperature, pH, re-use etc. different lipolytic enzymes may be utilized.

In the present application production of fatty acid alkyl esters have been tested by two different lipolytic enzymes Thermomyces lanuginosa lipase (TLL) and Candida antarctica B lipase (CALB) using ethanol (EtOH) and 2-propanol (iPrOH) respectively. The results are shown in examples 3 and 5 for TLL and examples 6 and 7 for CALB. Lipolytic activity of these enzymes is not identical which is in line with the results reported previously. One common feature is however apparent from these tests, namely the high conversion of fatty acid alkyl ester over 10 reaction cycles when using a molar ratio of at least 3.0 equivalents alcohol to fatty acids in the substrate. It seems that CALB maintain some activity at a molar ratio of 2.0 equivalents, but the activity increases at a molar ratio of at least 3.0 equivalents.

In certain embodiments the present invention relates to a method of producing fatty acid ethyl esters, wherein the at least one immobilized lipolytic enzyme is selected from the group containing: Thermomyces lanuginosa lipase; Candida Antarctica A lipase; Candida Antarctica B lipase; Candida deformans lipase; Candida lipolytica lipase; Candida parapsilosis lipase; Candida rugosa lipase; Cryptococcus spp. S-2 lipase; Rhizomucor miehei lipase; Rhizomucor delemar lipase; Burkholderia (Pseudomonas) cepacia lipase; Pseudomonas camembertii lipase; Pseudomonas fluorescens lipase; Geotrichium candidum lipase; Hyphozyma sp. lipase; Klebsiella oxytoca lipase; and variants thereof.

In certain embodiments the present invention relates to a method of producing fatty acid ethyl esters, wherein the at least one immobilized lipolytic enzyme is at least 60%; at least 70%; at least 75%; at least 80%; at least 85%; at least 88%; at least 90%; at least 92%; at least 94%; at least 95%; at least 96%; at least 97%; at least 98%; or at least 99% identical to an enzyme selected from the group containing: Thermomyces lanuginosa lipase; Candida Antarctica A lipase; Candida Antarctica B lipase; Candida deformans lipase; Candida lipolytica lipase; Candida parapsilosis lipase; Candida rugosa lipase; Cryptococcus spp. S-2 lipase; Rhizomucor miehei lipase; Rhizomucor delemar lipase; Burkholderia (Pseudomonas) cepacia lipase; Pseudomonas camembertii lipase; Pseudomonas fluorescens lipase; Geotrichium candidum lipase; Hyphozyma sp. lipase; Klebsiella oxytoca lipase. The identity may be calculated based on either amino acid sequences or nucleotide sequences.

The relatedness between two amino acid sequences or between two nucleotide sequences is described by the parameter “identity”. For purposes of the present invention, the degree of identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends in Genetics 16: 276-277), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues×100)/(Length of Alignment−Total Number of Gaps in Alignment)

For purposes of the present invention, the degree of identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the—nobrief option) is used as the percent identity and is calculated as follows:

(Identical Deoxyribonucleotides×100)/(Length of Alignment−Total Number of Gaps in Alignment)

### Lipolytic Enzyme Loading

Enzyme loading is for the purpose of the present invention expressed as the percentage weight/weight (% w/w) of immobilized lipolytic enzyme (enzyme+support material) present in the reaction mixture with respect to the substrate. Although an increased amount of lipolytic enzyme in general reduces the conversion time it is desirable from an economic point of view to operate at reduced levels of enzyme loading.

In certain embodiments the present invention relates to a method of producing fatty acid ethyl esters, wherein the at least one immobilized lipolytic enzyme loading is below 25.0% w/w; below 22.5% w/w; below 20.0% w/w; below 17.5% w/w; below 15.0% w/w; below 12.5% w/w; below 10.0% w/w; below 7.5% w/w; below 5.0% w/w; or below 2.5% w/w with respect to the substrate.

### Immobilization of Lipolytic Enzymes

The use of immobilized enzymes in oils and fats processing are experiencing significant growth due to new technology developments that have enabled cost effective interesterification of triglycerides (to modify melting properties) for margarine and shortenings. A fundamental advantage of immobilized enzymes is that they can be recovered and re-used from a batch process by simple filtration. Further, packing of immobilized enzymes in columns allows for easy implementation of a continuous process. Immobilized enzymes generally also have a positive effect on operational stability of the catalyst (compared to free enzymes), it makes handling easier (compared to free enzyme powder), and it allows operation under low-water conditions (compared to liquid formulated enzymes).

In certain embodiments the present invention relates to a method of producing fatty acid ethyl esters, wherein the lipolytic enzyme is immobilized either on a carrier; by entrapment in natural or synthetic matrices, such as sol-gels, alginate, and carrageenan; by cross-linking methods such as in cross-linked enzyme crystals (CLEC) and cross-linked enzyme aggregates (CLEA); or by precipitation on salt crystals such as protein-coated micro-crystals (PCMC).

In certain embodiments the present invention relates to a method of producing fatty acid ethyl esters, wherein the carrier is a hydrophilic carrier selected from the group containing: porous in-organic particles composed of alumina, silica and silicates such as porous glass, zeolites, diatomaceous earth, bentonite, vermiculite, hydrotalcite; and porous organic particles composed of carbohydrate polymers such as agarose or cellulose.

It is well-know that the nature of the carrier may have a very significant effect on the properties of the immobilized enzyme. Two commonly applied commercial enzymes, Novozym 435 and Lipozyme TL IM represent examples of a hydrophobic carrier (Novozym 435) and a hydrophilic carrier (Lipozyme TL IM). Hydrophilic carriers are often preferred over hydrophobic polymeric resins from a cost-perspective, but their properties can prevent their utilization in certain applications.

### Molar Ratio of Ethanol to Fatty Acid in the Substrate (EtOH: FA)

Excess of alcohol may drive the equilibrium reaction towards full conversion. For the purpose of the present invention the amount of alcohol is stated in equivalents (eq.) that is molar ratio of ethanol to fatty acid present in the substrate (EtOH:FA).

In certain embodiments the present invention relates to a method of producing fatty acid ethyl esters, wherein the molar ratio of ethanol to fatty acid in the substrate (EtOH:FA) is at least 3.5; 4.0; 4.5; 5.0; 5.5; 6.0; 6.5; 7.0; 7.5; 8.0; 8.5; 9.0; 9.5 or 10.0 equivalents.

Proteins are in general unstable in the presence of short-chain alcohols such as methanol and ethanol and inactivation of lipolytic enzymes occurs rapidly upon contact with insoluble alcohol, which exists as drops in the oil. Accordingly, it is often recommended that the amount of alcohol is kept below its solubility limits in oil. This may be obtained by a continuous or step-wise addition of alcohol.

In certain embodiments the present invention relates to a method of producing fatty acid ethyl esters, wherein ethanol is added continuous or step-wise.

Depending on the total amount of ethanol to be used in the conversion reaction the number of steps in step-wise addition may vary. Thus, step-wise addition may constitute at least 2 steps; at least 3 steps; at least 4 steps; at least 5 steps; at least 6 steps; at least 7 steps; at least 8 steps; at least 9 steps; or at least 10 steps.

### Enzymatic Biodiesel Process Design

The process setup is very important as it has to take into account technical issues, such as homogeneity of reaction/product mixture, solubility of alcohol, stability of enzyme, recovery of enzyme, etc. There are several different process designs to be considered: batch, continuous stirred tank reactors and packed bed reactors. These will briefly be outlined in the following paragraphs.

The batch process is a typical process used in the laboratory due to the simple setup. This process can be operated with addition of all components from the start, i.e. in bulk, or with step-wise addition of alcohol which is recommended. The batch process is useful in collecting data about the process, as for instance productivity of the enzyme. Negative elements of this process setup in large scale are the large tank volume required, the long reaction time, and the fact that this process is not continuous. Another very important fact to consider is the gradual decline in enzyme activity as the number of re-uses increase. When the enzyme activity decreases, the reaction time must be increased accordingly to keep a constant degree of conversion. With time, the capacity of the plant will decrease and eventually become unacceptable low. This is the time when the enzyme must be replaced. Though, the difficult decision is the compromise between capacity and cost of catalyst.

A continuous stirred tank reactor is a container with a continuous supply of feed and withdrawal of product. The design requires multiple tanks in series to assure the same degree of conversion for the same reaction time, meaning the total tank volume will also be large. The advantage of such system is that the capacity of the plant can be more constant as the tanks can hold enzymes of different age/activity. This also implies that the enzyme can be used more effectively until the activity has become very low. Another advantage of this design is the possibility of introducing separation steps between the tanks such as to eliminate the glycerol formed.

A system of packed bed columns with immobilized enzymes results in a well defined contact time between the liquid reactants and the solid catalyst. Furthermore, with this setup the enzyme to substrate ratio will be high at any specific time, and the whole system can be designed to be relatively compact. Commercial scale precedence for this technology already exists for enzymatic interesterification of oils. For enzymatic biodiesel production the issue with inactivation of the enzyme by addition of alcohol in concentrations higher than the solubility may be solved by step-wise addition before each column. In a similar way, the glycerol produced in the reaction may be removed between the columns.

In certain embodiments the present invention relates to a method of producing fatty acid ethyl esters, wherein said method is selected from the group of process designs containing: batch, continuous stirred-tank reactor, packed-bed column, and expanded-bed reactor.

### Feed Stocks for Enzymatic Production of Biodiesel

Fatty acid ethyl esters may be prepared from several types of vegetable oils. In the global vegetable oil production palm oil is leading the gains and has the highest yield compared to that of other vegetable oils, and it would therefore be economically intuitive to consider palm oil as a favorable feed stock for biodiesel production. One may, however, argue in favor of using inedible oils such as Jatropha oil, as edible oils are not in surplus supply. Examples of plants which may serve as feed stock for vegetable oils for use as substrate in the production of fatty acid ethyl esters are such as babassu, borage, canola, coconut, corn, cotton, hemp, jatropha, karanj, mustard, palm, peanut, rapeseed, rice, soybean, and sunflower.

Microalgae is also considered as feed stock in the production of biodiesel due to the higher photosynthetic efficiency of microalgae in comparison with plants and hence a potentially higher productivity per unit area.

Alternatively, fatty acid ethyl esters may be prepared from non-vegetable feed stocks like animal fat such as lard, tallow, butterfat and poultry; or marine oils such as tuna oil and hoki liver oil.

It has been estimated that 60-90% of the biodiesel cost arises from the cost of the feed stock oil, and thus use of cheaper waste oil would have a great impact in reducing the cost of biodiesel. In addition, it is considered an important step in reducing and recycling waste oil. Fresh vegetable oil and its waste differ in their content of water and free fatty acid. Unlike the conventional chemical routes for synthesis of diesel fuels, biocatalytic routes permit one to carry out the transesterification of a wide variety of oil feed stocks in the presence of acidic impurities, such as free fatty acids. Accordingly, fatty acid distillates (from deodorizer/fatty acid stripping), acid oils (from soap stock splitting in chemical oil refining), waste oils and used oils may serve as feed stock in the production of biodiesel.

Thus, the feed stock can be of crude quality or further processed (refined, bleached and deodorized). Suitable oils and fats may be pure triglyceride or a mixture of triglyceride, diglyceride, monoglyceride, and free fatty acids, commonly seen in waste vegetable oil and animal fats. The feed stock may also be obtained from vegetable oil deodorizer distillates. The type of fatty acids in the feed stock comprises those naturally occurring as glycerides in vegetable and animal fats and oils. These include oleic acid, linoleic acid, linolenic acid, palmetic acid and lauric acid to name a few. Minor constituents in crude vegetable oils are typically phospholipids, free fatty acids and partial glycerides i.e. mono- and diglycerides.

In certain embodiments the present invention relates to a method of producing fatty acid ethyl esters, wherein the substrate is selected from the group containing: babassu oil; borage oil; canola oil; coconut oil; corn oil; cotton oil; hemp oil; jatropha oil; karanj oil; mustard oil; palm oil; peanut oil; rapeseed oil; rice oil; soybean oil; and sunflower oil; oil from microalgae; animal fat; tallow; lard; butterfat; poultry; marine oils; tuna oil; hoki liver oil; fatty acid distillates; acid oils; waste oil; used oil; partial glycerides and any combinations thereof.

### Re-Use of Immobilized Lipolytic Enzyme in the Production of Fatty Acid Ethyl Esters

In certain embodiments the present invention relates to re-use of at least one lipolytic enzyme immobilized on a hydrophilic carrier in the production of fatty acid ethyl esters obtained by reacting ethanol with a substrate comprising triglyceride, diglyceride, monoglyceride; free fatty acids or any combination thereof, wherein the molar ratio of ethanol to fatty acid in the substrate (EtOH:FA) is at least 3.0 equivalents; the enzyme loading is below 30% w/w with respect to the substrate; and which enzyme after use in a conversion reaction is separated from the resulting reaction mixture and re-used directly without modifications in the next conversion reaction. By modification is meant any treatment or activity such as activation, washing, drying etc. apart from the separation of the immobilized lipolytic enzyme from the reaction mixture.

In certain embodiments the present invention relates to re-use of at least one immobilized lipolytic enzyme in the production of fatty acid ethyl esters, wherein the immobilized lipolytic enzyme is selected from the group containing: Thermomyces lanuginosa lipase; Candida Antarctica A lipase; Candida Antarctica B lipase; Candida deformans lipase; Candida lipolytica lipase; Candida parapsilosis lipase; Candida rugosa lipase; Cryptococcus spp. S-2 lipase; Rhizomucor miehei lipase; Rhizomucor delemar lipase; Burkholderia (Pseudomonas) cepacia lipase; Pseudomonas camembertii lipase; Pseudomonas fluorescens lipase; Geotrichium candidum lipase; Hyphozyma sp. lipase; Klebsiella oxytoca lipase; and variants thereof.

In certain embodiments the present invention relates to re-use of at least one immobilized lipolytic enzyme in the production of fatty acid ethyl esters, wherein the at least one immobilized lipolytic enzyme loading is below 25.0% w/w; below 22.5% w/w; below 20.0% w/w; below 17.5% w/w; below 15.0% w/w; below 12.5% w/w; below 10.0% w/w; below 7.5% w/w; below 5.0% w/w; or below 2.5% w/w with respect to the substrate.

In certain embodiments the present invention relates to re-use of at least one immobilized lipolytic enzyme in the production of fatty acid ethyl esters, wherein the lipolytic enzyme is immobilized either on a carrier; by entrapment in natural or synthetic matrices, such as sol-gels, alginate, and carrageenan; by cross-linking methods such as in cross-linked enzyme crystals (CLEC) and cross-linked enzyme aggregates (CLEA); or by precipitation on salt crystals such as protein-coated micro-crystals (PCMC).

In certain embodiments the present invention relates to re-use of at least one immobilized lipolytic enzyme in the production of fatty acid ethyl esters, wherein the carrier is a hydrophilic carrier selected from the group containing: porous in-organic particles composed of alumina, silica and silicates such as porous glass, zeolites, diatomaceous earth, bentonite, vermiculite, hydrotalcite; and porous organic particles composed of carbohydrate polymers such as agarose or cellulose.

In certain embodiments the present invention relates to re-use of at least one immobilized lipolytic enzyme in the production of fatty acid ethyl esters, wherein the molar ratio of ethanol to fatty acid in the substrate (EtOH:FA) is at least 3.5; 4.0; 4.5; 5.0; 5.5; 6.0; 6.5; 7.0; 7.5; 8.0; 8.5; 9.0; 9.5 or 10.0 equivalents.

In certain embodiments the present invention relates to re-use of at least one immobilized lipolytic enzyme in the production of fatty acid ethyl esters, wherein ethanol is added continuous or step-wise.

In certain embodiments the present invention relates to re-use of at least one immobilized lipolytic enzyme in the production of fatty acid ethyl esters, wherein said method is selected from the group of process designs containing: batch, continuous stirred-tank reactor, packed-bed column, and expanded-bed reactor.

In certain embodiments the present invention relates to re-use of at least one immobilized lipolytic enzyme in the production of fatty acid ethyl esters, wherein the substrate is selected from the group containing: babassu oil; borage oil; canola oil; coconut oil; corn oil; cotton oil; hemp oil; jatropha oil; karanj oil; mustard oil; palm oil; peanut oil; rapeseed oil; rice oil; soybean oil; and sunflower oil; oil from microalgae; animal fat; tallow; lard; butterfat; poultry; marine oils; tuna oil; hoki liver oil; fatty acid distillates; acid oils; waste oil; used oil; partial glycerides and any combinations thereof.

### Composition and its Use as Fuel

In certain embodiments the present invention relates to a composition obtained by the method of producing fatty acid ethyl esters, wherein said composition comprises at least two of the following components selected from the group containing: fatty acid ethyl esters; triglyceride; diglyceride; monoglyceride; glycerol; and water.

Fatty acid alkyl esters are used in an extensive range of products and as synthetic intermediates. Some of their industrial applications include use as lubricants, plasticizers, antirust agents, drilling and cutting oils, and starting materials for synthesis of superamides and fatty alcohols. Various fatty acid alkyl esters find use in cosmetics or as salad oil. Certain embodiments of the present invention in particular relates to fuels. Fatty acid alkyl esters of short-chain alcohols are non-toxic, biodegradable and an excellent replacement wholly or partly for petroleum based fuel due to the similarity in cetane number, energy content, viscosity and phase changes to those of petroleum based fuels.

According to certain embodiments the present invention relates to compositions consisting of a mixture of at least two of the following components: FAEE; triglyceride; diglyceride; monoglycerides; glycerol; and water. The composition may potentially be refined or purified by methods known in the art such as distillation (including flash evaporation, stripping, and deodorization); phase separation; extraction; and drying. The purpose of such refining could be to remove or recover one or more of the above mentioned components from the composition. Examples include, but are not limited to, drying for the removal of water; phase separation for the removal of glycerol; and distillation for the isolation of FAEE. Hence, it can be envisioned that the crude reaction mixture (composition) can be applied without further refining, or refined by one or more methods.

In certain embodiments the present invention relates to use of the composition obtained by the method of producing fatty acid ethyl esters as fuel.

In certain embodiments the present invention relates to use of the composition obtained by the method of producing fatty acid ethyl esters as fuel, wherein the composition is refined.

In certain embodiments the present invention relates to a fuel comprising the composition obtained by the method of producing fatty acid ethyl esters.

In certain embodiments the present invention relates to a fuel comprising the composition obtained by the method of producing fatty acid ethyl esters, wherein the composition is refined.

The present invention is further described by the following examples that should not be construed as limiting the scope of the invention.

### EXAMPLES

Chemicals used as buffers and substrates were commercial products of at least reagent grade.

### Media and Solutions

Refined, bleached, deodorized soybean oil (SBO) was purchased from Hørkram Schulz Food Service NS (Horning, Denmark). Ethanol was absolute (99.8% v/v) purchased from Sigma-Aldrich. All other chemicals were purchased from Sigma-Aldrich and used without further purification. NMR analyses were performed on a Varian Mercury VX-400 MHz system at 30° C. using CDCl₃ solvent. Thermomyces lanuginosa lipase (TLL) immobilized on silica and Candida antarctica B lipase (CALB) immobilized on silica were obtained from Novozymes NS, Bagsvrd, Denmark.

### Example 1

### Re-use of Immobilized Thermomyces lanuginosa Lipase in Batch Synthesis of FAEE Using 1-6 eq. EtOH

We studied the synthesis of fatty acid ethyl esters using soybean oil (SBO) and ethanol (EtOH) in a reaction catalyzed by Thermomyces lanuginosa lipase (TLL) immobilized on silica (Novozymes A/S, Bagsvrd, Denmark). The FAEE reactions were performed in 100 mL screwcap conical flasks. 20 mL SBO and 1 g immobilized enzyme were added to each flask. The amount of EtOH added varied from 1 to 6 molar equivalents relative to the total amount of fatty acids in the oil (i.e. [EtOH]:[FA]). EtOH was added step-wise in three equal portions at t=0 h, t=2 h, and t=4 h. To initiate the reactions, the first portion of EtOH was added and the flasks were closed and placed in a water bath orbital shaker at 35° C.

The conversion to FAEE was followed by ¹H NMR analysis. Hence, aliquots of 20 microliter were withdrawn from the reaction mixture for analysis after 4 h and after 24 h where the reaction is at equilibrium. Conversions to FAEE were calculated as described in “Quantification of soybean oil ethanolysis with ¹H NMR” Neto et al. (2004) J. Am. Oil Chem. Soc. Vol. 81, p. 1111-1114.

After 24 h all reactions were terminated by decanting the reaction mixture from the immobilized enzyme. Another reaction cycle was immediately initiated by adding new SBO and EtOH to the immobilized enzyme.

TABLE 1
Content of FAEE (% w/w) after 4 h/24 h.

| | EtOH | | | | | |
|---|---|---|---|---|---|---|
| Cycle no. | 1 eq. | 2 eq. | 3 eq. | 4 eq. | 5 eq. | 6 eq. |
| 1 | 49/66 | 67/88 | 63/97 | 49/97 | 37/93 | 35/93 |
| 2 | 4/4 | 4/4 | 25/89 | 39/88 | 36/95 | 31/96 |
| 3 | 3/3 | 3/3 | 23/89 | 42/95 | 38/95 | 29/94 |
| 4 | 3/3 | 3/3 | 11/82 | 30/94 | 31/96 | 29/91 |
| 5 | 3/3 | 3/3 | 24/85 | 42/93 | 49/95 | 51/97 |

| | | | | | |
|---|---|---|---|---|---|
| 6 | Reactions discontinued | 18/87 | 34/92 | 46/95 | 40/97 |
| 7 | | 12/- | 35/- | 50/- | 61/- |
| 8 | | 9/61 | 21/88 | 35/95 | 39/92 |
| 9 | | 60/83 | 67/93 | 73/95 | 78/97 |
| 10 | | 60/81 | 67/94 | 71/96 | 70/96 |

The above data clearly documents that the enzyme is inactivated in the presence of 1 eq. or 2 eq. EtOH, whereas in reactions with 3 to 6 eq. EtOH, enzyme activity is preserved through at least 10 cycles.

### Example 2

### Re-Use of Immobilized Thermomyces lanuginosa Lipase in Batch Synthesis of FAEE with a Solvent Wash Between Cycles

This experiment was conducted essentially as the experiment described in Example 1 with the following amendments. Only 2.0 eq. and 3.5 eq. EtOH were tested. The EtOH was added step-wise at t=0 h: 0.5 eq.; t=2 h: 0.5 eq.; t=4 h: 1 eq. (for at total of 2 eq.) or at t=0 h: 1 eq.; t=2 h: 1 eq.; t=4 h: 1.5 eq. (for a total of 3.5 eq.). After each cycle, the reaction mixtures were decanted from the immobilized enzyme and submitted to the following treatments: a) no wash; b) wash with hexane; or c) wash with tert-butanol (t-BuOH). After the treatment, new SBO and EtOH were added and the next reaction cycle initiated (i.e. no attempt to remove residual solvent by drying the enzyme). Samples for NMR analysis were taken after 6 h and 24 h.

TABLE 2
Content of FAEE (% w/w) using 2 eq. EtOH

| | No wash | | Hexane wash | | t-BuOH wash | |
|---|---|---|---|---|---|---|
| Cycle no. | 6 h | 24 h | 6 h | 24 h | 6 h | 24 h |
| 1 | 87 | 100 | 85 | 86 | 76 | 87 |
| 2 | 8 | 8 | 5 | 4 | 0 | 5 |
| 3 | 2 | 4 | 2 | 4 | 2 | 3 |
| 4 | 1 | 2 | 1 | 2 | 1 | 2 |

TABLE 3
Content of FAEE (% w/w) using 3.5 eq. EtOH

| | No washing | | Hexane wash | | t-BuOH wash | |
|---|---|---|---|---|---|---|
| Cycle no. | 6 h | 24 h | 6 h | 24 h | 6 h | 24 h |
| 1 | 100 | 100 | 100 | 100 | 100 | 100 |
| 2 | 63 | 100 | 66 | 100 | 60 | 100 |
| 3 | 58 | 100 | 61 | 100 | 47 | 100 |
| 4 | 49 | 100 | 61 | 100 | 41 | 100 |
| 5 | 57 | 100 | 61 | 100 | 40 | 100 |
| 6 | 73 | 100 | 63 | 100 | 41 | 100 |
| 7 | 68 | 100 | 60 | 100 | 44 | 100 |
| 8 | 76 | 100 | 82 | 100 | 68 | 95 |
| 9 | 77 | 100 | 95 | 100 | 56 | 100 |
| 10 | 75 | 100 | 99 | 100 | 63 | 100 |

These data show that the enzyme is quickly inactivated with 2 eq. EtOH, even if the enzyme is washed with hexane or t-BuOH between cycles. With 3.5 eq. EtOH, enzymatic activity is preserved through the 10 cycles tested here, regardless of whether the enzyme is washed or not.

### Example 3

### Re-Use of Immobilized Thermomyces lanuginosa Lipase in Batch Synthesis of FAEE with One-Step/Bulk Addition of EtOH

This experiment was conducted essentially as the experiment described in Example 1 with the following amendment. EtOH was not added step-wise but added in bulk at t=0 h.

TABLE 4
Content of FAEE (% w/w) after 4 h/24 h.

| | EtOH | | | | | |
|---|---|---|---|---|---|---|
| Cycle no. | 1 eq. | 2 eq. | 3 eq. | 4 eq. | 5 eq. | 6 eq. |
| 1 | 63/72 | 17/86 | 10/82 | 7/74 | 8/65 | 5/62 |
| 2 | 4/4 | 5/10 | 3/67 | 6/57 | 4/49 | 5/42 |
| 3 | 3/3 | 3/5 | 5/63 | 5/39 | 4/32 | 3/37 |
| 4 | 3/3 | 4/6 | 5/31 | 6/36 | 5/26 | 3/22 |
| 5 | 3/3 | 3/4 | 3/29 | 3/29 | 3/19 | 3/17 |
| 6 | 3/3 | 3/3 | 3/20 | 4/23 | 3/19 | 3/12 |
| 7 | 3/3 | 3/3 | 3/18 | 3/19 | 3/12 | 3/10 |
| 8 | | 4/5 | 5/21 | 4/17 | 4/11 | 3/10 |
| 9 | | 3/11 | 4/36 | 4/36 | 3/29 | 3/46 |
| 10 | | -/- | 6/33 | 5/29 | 4/26 | 3/20 |

The results show that below 3.0 eq. EtOH, enzymatic activity is quickly lost and at least 3 eq. EtOH some enzymatic activity is preserved through the 10 cycles. In comparison with a step-wise addition as described in example 1 the one-step or bulk addition of EtOH is clearly detrimental for the enzyme. Accordingly, a step-wise or alternatively a continuous addition of EtOH is preferred.

### Example 4

### Re-Use of Immobilized Thermomyces lanuginosa Lipase in Batch Synthesis of FAME Using 1-6 eq. MeOH

This experiment was conducted essentially as the experiment described in Example 1 with the following amendments. 1-6 eq. methanol (MeOH) was added instead of EtOH (i.e. FAME synthesis).

Conversion was calculated from the ¹H NMR spectra as % FAME=100*(A2/3)/(A3/3), with A2 being the integral of —OCH₃ [3.60-3.70 ppm] and A3 being the integral of —CH₃ from all fatty acids [0.85-0.95 ppm].

TABLE 5
FAME content (% w/w) after 4 h/24 h.

| | MeOH | | | | | |
|---|---|---|---|---|---|---|
| Cycle no. | 1 eq. | 2 eq. | 3 eq. | 4 eq. | 5 eq. | 6 eq. |
| 1 | 46/48 | 1/0 | 1/1 | 1/1 | 1/2 | 1/1 |
| 2 | 4/5 | 0/1 | 0/0 | 0/0 | 0/0 | 0/0 |
| 3 | 1/- | 0/- | 0/- | 0/- | 0/- | 0/- |

Only reaction 1 (with 1.0 eq. MeOH) showed some conversion in the first cycle. This activity was markedly decreased in cycle no. 2 and absent in cycle no, 3. Hence, the result of a high production of FAEE in the presence of at least 3.0 eq. EtOH was not observed when using MeOH for FAME synthesis.

### Example 5

### Re-Use of Immobilized Thermomyces lanuginosa Lipase in Batch Synthesis of FAIE Using 1-6 eq. iPrOH

This experiment was conducted essentially as the experiment described in Example 1 with the following amendments. 1-6 eq. 2-propanol (iPrOH) was used instead of EtOH (i.e. FAIE synthesis). iPrOH was added in one portion at t=0 h.

Conversion was calculated from the ¹H NMR spectra as % FAIE=100*(A2/1)/(A3/3), with A2 being the integral of —OCH(CH₃)₂ [5.00-5.05 ppm] and A3 being the integral of —CH₃ from all fatty acids [0.85-0.95 ppm].

In this experiment, no conversion to FAIE could be observed in any of the reactions. This is probably because the TLL enzyme does not accept iPrOH in its active site and may thus not catalyze this reaction.

### Example 6

### Re-Use of Candida antarctica B Lipase in Batch Synthesis of FAEE Using 1-6 eq. EtOH

This experiment was conducted essentially as the experiment described in Example 1 with the following amendments. Candida antarctica B-lipase (CALB) immobilized on silica (Novozymes A/S, Bagsværd, Denmark) was used instead of Thermomyces lanuginosa lipase. EtOH was not added step-wise but added in bulk at t=0 h.

TABLE 6
FAEE content (% w/w) after 4 h/24 h.

| | EtOH | | | | | |
|---|---|---|---|---|---|---|
| Cycle no. | 1 eq. | 2 eq. | 3 eq. | 4 eq. | 5 eq. | 6 eq. |
| 1 | 45/67 | 43/97 | 40/97 | 58/97 | 54/97 | 42/97 |
| 2 | 10/13 | 13/55 | 49/97 | 92/97 | 81/97 | 60/97 |
| 3 | 4/11 | 14/50 | 26/97 | 58/97 | 85/97 | 59/97 |
| 4 | 3/7 | 5/48 | 15/84 | 32/93 | 32/90 | 24/72 |
| 5 | 3/7 | 7/33 | 9/74 | 19/92 | 19/93 | 13/90 |
| 6 | 3/6 | 5/37 | 7/66 | 16/13 | 15/94 | 12/93 |
| 7 | 3/6 | 4/39 | 5/74 | 14/93 | 17/93 | 12/92 |
| 8 | 3/6 | 4/42 | 6/72 | 12/85 | 15/93 | 13/84 |
| 9 | 4/6 | 6/31 | 7/53 | 15/80 | 18/89 | 11/80 |
| 10 | 3/5 | 4/35 | 5/53 | 12/87 | 21/91 | 12/84 |

The data illustrates that 1 eq. and 2 eq. EtOH results in inferior results. With 1 eq. EtOH, the enzyme is inactivated after the third cycle. With 2 eq. EtOH, some activity is maintained all through 10 cycles. This is in contrast to the results with TLL, in which reactions with 1 eq. and 2 eq. EtOH resulted in enzyme inactivation already after cycle no. 1. Hence, in this example, the optimal dosage of EtOH seems to be approx. 4-5 eq.

### Example 7

### Re-Use of Candida antarctica B Lipase in Batch Synthesis of FAIE Using 1-6 eq. EtOH

This experiment was conducted essentially as the experiment described in Example 1 with the following amendments. Candida antarctica B lipase (CALB) immobilized on silica (, Novozymes NS, Bagsværd, Denmark) was used instead of Thermomyces lanuginosa lipase. 2-propanol (iPrOH) was used instead of EtOH (i.e. FAIE synthesis). iPrOH was added in one portion at t=0 h.

Conversion was calculated from the ¹H NMR spectra as % FAIE=100*(A2/1)/(A3/3), with A2 being the integral of —OCH(CH₃)₂ [5.00-5.05 ppm] and A3 being the integral of —CH₃ from all fatty acids [0.85-0.95 ppm].

TABLE 7
FAIE content (% w/w) after 4 h/24 h.

| | iPrOH | | | | | |
|---|---|---|---|---|---|---|
| Cycle no. | 1 eq. | 2 eq. | 3 eq. | 4 eq. | 5 eq. | 6 eq. |
| 1 | 30/57 | 23/81 | 22/78 | 23/73 | 23/79 | 23/76 |
| 2 | 17/26 | 30/80 | 38/86 | 33/83 | 31/78 | 30/29 |
| 3 | 3/5 | 22/69 | 37/80 | 35/82 | 31/82 | 29/81 |
| 4 | 6/24 | 20/59 | 37/83 | 36/85 | 34/84 | 33/86 |
| 5 | 3/7 | 22/70 | 38/83 | 38/86 | 34/84 | 33/84 |
| 6 | 9/32 | 22/72 | 37/83 | 39/86 | 35/86 | 33/83 |
| 7 | 3/6 | 24/73 | 42/86 | 39/87 | 37/86 | 35/87 |
| 8 | 6/31 | 23/72 | 40/86 | 40/88 | 36/88 | 35/87 |
| 9 | 3/6 | 25/73 | 40/87 | 42/88 | 36/89 | 36/90 |
| 10 | 5/33 | 22/71 | 37/86 | 38/90 | 34/88 | 33/85 |

Clearly, CALB may in contrast to TLL catalyze this reaction (compare with Example 5). High conversions (70-90%) are obtained in most reactions after 24 h. Again at least 3.0 eq. alcohol results in higher conversions (compared to the reactions with 1.0 eq. or 2.0 eq. EtOH).

1-15. (canceled)

16. A method of producing fatty acid ethyl esters comprising:
a) reacting a substrate comprising triglycerides, diglycerides, monoglycerides, free fatty acids, or any combination thereof, with at least one immobilized lipolytic enzyme, to provide a reaction mixture wherein the enzyme loading is below 30% w/w with respect to the substrate, and the molar ratio of ethanol to fatty acid (EtOH:FA) is at least 3.0 equivalents;
b) separating the immobilized lipolytic enzyme from the resulting reaction mixture; and
c) subjecting the immobilized lipolytic enzyme to at least one further reaction directly without modifications.

17. The method of claim 16, wherein the immobilized lipolytic enzyme is selected from the group consisting of: Thermomyces lanuginosa lipase; Candida Antarctica A lipase; Candida Antarctica B lipase; Candida deformans lipase; Candida lipolytica lipase; Candida parapsilosis lipase; Candida rugosa lipase; Cryptococcus spp. S-2 lipase; Rhizomucor miehei lipase; Rhizomucor delemar lipase; Burkholderia (Pseudomonas) cepacia lipase; Pseudomonas camembertii lipase; Pseudomonas fluorescens lipase; Geotrichium candidum lipase; Hyphozyma sp. lipase; Klebsiella oxytoca lipase; and variants thereof.

18. The method of claim 16, wherein the at least one immobilized lipolytic enzyme loading is below 25.0% w/w with respect to the substrate.

19. The method of claim 16, wherein the lipolytic enzyme is immobilized either on a carrier; by entrapment in natural or synthetic matrices; by cross-linking methods; or by precipitation on salt crystals.

20. The method of claim 19, wherein the carrier is a hydrophilic carrier selected from the group containing: porous in-organic particles composed of alumina, silica and silicates such as porous glass, zeolites, diatomaceous earth, bentonite, vermiculite, hydrotalcite; and porous organic particles composed of carbohydrate polymers such as agarose or cellulose.

21. The method of claim 16, wherein the molar ratio of ethanol to fatty acid in the substrate (EtOH:FA) is at least 3.5 equivalents.

22. The method of claim 21, wherein ethanol is added continuous or step-wise.

23. The method of claim 16, wherein said method is selected from the group of process designs consisting of batch, continuous stirred-tank reactor, packed-bed column, and expanded-bed reactor.

24. The method of claim 16, wherein the substrate is selected from the group consisting of babassu oil; borage oil; canola oil; coconut oil; corn oil; cotton oil; hemp oil; jatropha oil; karanj oil; mustard oil; palm oil; peanut oil; rapeseed oil; rice oil; soybean oil;
and sunflower oil; oil from microalgae; animal fat; tallow; lard; butterfat; poultry; marine oils; tuna oil; hoki liver oil; fatty acid distillates; acid oils; waste oil; used oil; partial glycerides and any combinations thereof.

25. Re-use of at least one lipolytic enzyme immobilized on a hydrophilic carrier in the production of fatty acid ethyl esters obtained by reacting ethanol with a substrate comprising triglyceride, diglyceride, monoglyceride; free fatty acids or any combination thereof, wherein the molar ratio of ethanol to fatty acid in the substrate (EtOH:FA) is at least 3.0 equivalents; the enzyme loading is below 30% w/w with respect to the substrate; and which enzyme after use in a conversion reaction is separated from the resulting reaction mixture and re-used directly without modifications in the next conversion reaction.

26. A composition obtained by the method of claim 16, wherein said composition comprises at least two of the following components selected from the group consisting of fatty acid ethyl esters; triglyceride; diglyceride; monoglyceride; glycerol; and water.

27. A fuel comprising the composition obtained by the method of claim 16.

28. The fuel of claim 27 wherein the composition is refined.
